(19) United States
(12) Patent Application Publication
(10) Pub. No.: US 2011/0178290 A1
(43) Pub. Date: Jul. 21, 2011

(54) PROCESS FOR THE PRODUCTION OF HCL GAS FROM CHLORIDE SALTS AND FOR THE PRODUCTION OF CARBOHYDRATES

(75) Inventors: Avram BANIEL, Jerusalem (IL); Aharon EYAL, Jerusalem (IL)

(73) Assignee: HCL CLEANTECH LTD., TeI Aviv (IL)

(21) Appl. No.: 13/036,717

(22) Filed: Feb. 28, 2011

Related U.S. Application Data
(63) Continuation of application No. PCT/IL2009/000843, filed on Sep. 1, 2009.

(30) Foreign Application Priority Data
Sep. 2, 2008 (IL) IL/193,826
Aug. 31, 2009 (IL) IL/200,646

(51) Int. Cl. C07H 1/06; C01B 7/01; C01B 25/30

(52) U.S. Cl. 536/127; 423/488; 423/313

(57) ABSTRACT

A process for the co-production of gaseous HCl and a salt product comprising a cation and an anion, which process comprises the steps of a. providing an aqueous solution comprising protons, chloride anions, and cations and anions of the salt product, b. bringing the solution into contact with a substantially immiscible extractant, the extractant comprising: 1) an oil soluble amine, which amine is substantially water insoluble both in free and in salt form; and 2) a carrier solvent for the amine; whereupon HCl selectively transfers to the extractant to form an HCl-carrying extractant and a chloride depleted aqueous solution containing the salt product; c. separating the HCl-carrying extractant from the chloride-depleted aqueous solution; and d. distilling HCl from the separated HCl-carrying extractant to form gaseous HCl and HCl depleted extractant.

### CROSS-REFERENCE TO RELATED APPLICATIONS

This application is a continuation of PCT International Application Serial No. PCT/IL2009/000843 filed on Sep. 1, 2009, which claims the benefit of Israeli Patent Application Nos. 193,826 filed on Sep. 2, 2008, and 200,646 filed on Aug. 31, 2009, the contents of each of which are incorporated herein by reference.

### FIELD OF THE INVENTION

The present invention relates to a process for the production of HCl gas from a chloride salt. More particularly, the present invention relates to a process for the co-production of gaseous HCl and a product salt from chloride salt, e.g., an alkali or ammonium chloride salt, and acids other than hydrochloride acid or acidic salts of such acid as well as to a process for the production of carbohydrates.

### BACKGROUND OF THE INVENTION

As used in the present application HCl denotes hydrogen chloride. HCl by itself forms a gas phase and is commonly described as “HCl gas”.

Alkali chlorides, primarily sodium chloride NaCl and potassium chloride KCl, are the primary source chemicals for making alkali hydroxides, NaOH and KOH, and the corresponding bicarbonates and carbonates. These, in turn, are converted to an immense variety of inorganic and organic salts by neutralization with the corresponding inorganic or organic acids.

In the particular case of sodium sulfate Na₂SO₄ and potassium sulfate K₂SO₄, it is possible to react the chloride directly with sulfuric acid H₂SO₄, at high temperature, to obtain the sulfate and hydrogen chloride HCl according to the general reaction:

2MCl+H₂SO₄→M₂SO₄+2HCl_{gas}

wherein M denotes sodium Na or potassium K. In this particular case prior conversion of alkali chlorides to hydroxides or carbonates, by electrolysis or by reaction with lime, is profitably eliminated.

Such direct conversion to alkali salt and HCl is, however, restricted in practice to sulfuric acid that has the low volatility and the thermal stability required for reactions at high temperature.

### SUMMARY OF THE INVENTION

It was surprisingly found that the aim of direct conversion of alkali chlorides, which denoted MCl, to salts of inorganic or organic acids, which acids are denoted HX, can be achieved for a wide variety of acids that satisfy the conditions of being of weaker acidity than hydrochloric acid, as measured by a higher pKa value, and/or of higher hydrophilicity than hydrochloric acid, as measured, e.g. by higher heat of dissolution, and of having some water solubility. A number of such weaker acids and their pKa values are tabulated below in the order of decreasing acid strength, by way of illustration:

TABLE 1

| Acid HX | pKa values |
|---|---|
| Hydrochloric acid, HCl | −6.1 |
| Sulfuric acid, H₂SO₄ | −3; 1.96 |
| Nitric acid, HNO₃ | −1.44 |
| Phosphoric acid, H₃PO₄ | 2.15; 7.2; 12.35 |
| Chloro-acetic acid, ClCH₂COOH | 2.86 |
| Acetic acid, CH₃COOH | 4.76 |
| Carbonic acid, H₂CO₃ | 6.35 |

This conversion takes place by contacting an aqueous solution of the chosen chloride salt and of the chosen acid and/or an acidic salt of such acid with an amine extractant that selectively extracts HCl whereby the desired product salt is formed as an aqueous solution from which it is optionally recovered by methods known per-se and wherein the HCl-carrying extractant is heated to distill-off HCl gas.

### DETAILED DESCRIPTION OF THE INVENTION

Thus according to the present invention, there is now provided a process for the co-production of a gaseous HCl and a salt product comprising a cation and an anion, which process comprises the steps of: a. providing an aqueous solution comprising protons, chloride anions, and cations and anions of said salt product, b. bringing said solution into contact with a substantially immiscible extractant, said extractant comprising:

1) an oil soluble amine, which amine is substantially water insoluble both in free and in salt form; and 2) a carrier solvent for the amine;

whereupon HCl selectively transfers to said extractant to form an HCl-carrying extractant and a chloride depleted aqueous solution containing said salt product; c. separating said HCl-carrying extractant from said chloride-depleted aqueous solution; and d. distilling HCl from said separated HCl-carrying extractant to form gaseous HCl and HCl depleted extractant.

Preferably said extractant further comprises an oil soluble organic acid, which acid is substantially water insoluble both in free and in salt form.

Preferably, the pKa of the organic acid is above 3.

In preferred embodiments of the present invention said extractant is characterized by a pHhn of less than 3.

Preferably said process further comprises: e. recovering said salt product of said separated chloride-depleted aqueous solution of step (b).

In preferred embodiments of the present invention, step (a) is carried out by combining a chloride salt of said cation with a compound selected from an acid of said anion and an acidic salt of said anion in the presence of water.

In some preferred embodiments of the present invention said recovery comprises crystallization to form crystals of said salt and a mother liquor.

In some preferred embodiments, the mother liquor is preferably used for said providing of step (a).

In especially preferred embodiments of the present invention, said cation is selected from the group consisting of alkali ions and ammonium.

According to some embodiments, said anion is selected from the group consisting of anions of acids that are weaker than HCl and anions that are more hydrophilic than chloride anions.

In preferred embodiments of the present invention said anion is selected from the group consisting of phosphate, nitrate, sulfate, carbonate and bicarbonate.

Preferably said distilling is at a temperature lower than 250° C.

Preferably, said gaseous HCl comprises at least 90% of the chloride in said provided solution of step (a).

In especially preferred embodiments of the present invention there is provided a process for the production of gaseous HCl from an alkali or ammonium chloride salt comprising: a. combining an alkali or ammonium salt in solution with a water soluble acid or acidic salt having an acidity weaker than hydrochloric acid; b. bringing said solution into contact with a substantially immiscible extractant, said extractant comprising:

1) an oil soluble amine, which amine is substantially water insoluble both in free and in salt form;

2) an oil soluble weak organic acid having a pKa above 3, which acid is substantially water insoluble both in free and in salt form; and

3) a carrier solvent for the amine and organic acid; whereupon HCl selectively transfers to said extractant to form an HCl-carrying extractant and a chloride depleted solution containing the anion of said weak acid and the alkali or ammonium cation c. distilling HCl from said HCl-carrying extractant to form gaseous HCl and HCl depleted extractant.

In preferred embodiments of the present invention said process further comprises: d. recovering the salt of said anion of said weak acid and the alkali or ammonium cation from the chloride depleted solution of step (b).

In a preferred embodiment of the present invention phosphoric acid is combined in solution with potassium chloride and there is obtained substantially pure gaseous HCl and potassium phosphate.

Preferably in the aforementioned embodiments, said extractant is characterized by a pHhn of less than 3.

In especially preferred embodiments the pKa of the organic acid is above 3.

Thus it will be realized that for example, according to preferred embodiments of the present invention potassium chloride could be combined with sulfuric acid to produce gaseous HCl as well as to produce the commercially valuable fertilizer potassium sulfate and that potassium chloride could be combined with phosphoric acid to produce HCl as well as to produce potassium phosphate. Similarly, H₂SO₄ and NaCl could be used to produce HCl and Na₂SO₄.

The following combinations of salts and acids may be used illustratively to produce gaseous HCl and desirable salts as set forth below:

1. Combining H₃PO₄ and KCl to produce gaseous HCl and mono-potassium phosphate KH₂PO₄, di-potassium phosphate K₂HPO₄, and/or their combination

2. Combining KH₂PO₄ and KCl to produce gaseous HCl and di-potassium phosphate K₂HPO₄

3. Combining H₃PO₄ and NaCl to produce gaseous HCl and mono-sodium phosphate NaH₂PO₄, di-sodium phosphate Na₂HPO₄ or their combination

4. Combining H₃PO₄ and NH₄Cl to produce gaseous HCl and mono-ammonium phosphate NH₄H₂PO₄, di-ammonium phosphate (NH₄)₂HPO₄ and/or their combination

5. Combining NH₄H₂PO₄ and KCl to produce gaseous HCl and di-ammonium phosphate (NH₄)2HPO₄

6. Combining H₂CO₃ and KCl or (CO₂+H₂O) and KCl to produce gaseous HCl and KHCO₃

7. Combining H₂CO₃ and NaCl or (CO₂+H₂O) and NaCl to produce gaseous HCl and NaHCO₃

8. Combining H₂CO₃ and NH₄Cl or (CO₂+H₂O) and NH₄Cl to produce gaseous HCl and NH₄HCO₃

9. Combining Acetic acid and NaCl or acetic acid and KCl to produce gaseous HCl and sodium acetate or potassium acetate respectively.

Concentration of hydrochloric acid by distillation is a well-known technology practiced for many years. Its basic drawback is the high cost of the equipment and the inherent large energy consumption. If various impurities are present in the dilute hydrochloric acid, the concentration by distillation needs to be preceded by some separation step to prevent equipment fouling or contamination of the concentrated hydrochloric acid.

In U.S. Pat. No. 4,291,007 by one of the present inventors, there is described and claimed a solvent extraction process for the separation of a strong mineral acid from other species present in an aqueous solution and the recovery thereof under reversible conditions utilizing an extractant phase that contains an acid-base-couple (hereinafter referred to as an “ABC extractant”) which obviates the consumption of chemicals for regeneration, comprising the steps of: a) bringing an aqueous solution containing the mineral acid to be separated into contact with a substantially immiscible extractant phase, said extractant phase comprising:

1) a strong organic acid, which acid is oil-soluble and substantially water-immiscible, in both free and salt forms;

2) an oil-soluble amine, which amine is substantially water-insoluble, in both free and salt forms; and

3) a carrier solvent for said organic acid and said amine, wherein the molar ratio of said organic acid to said amine is between about 0.5:2 and 2:0.5, whereupon said predetermined mineral acid selectively and reversibly transfers to said extractant phase; b) separating said two phases; and c) backwashing said extractant phase with an aqueous system to recover substantially all the mineral acid contained in said extractant phase.

The strong organic acids envisioned for use in the extractant phase of said invention were organic acids which may be defined and characterized as follows: When 1 mol of the acid in a 0.2 molar or higher concentration is contacted with an equivalent amount of 1N NaCl, the pH of the sodium chloride solution decreases to below 3.

Especially preferred for use in said invention were strong organic acids selected from the group consisting of aliphatic and aromatic sulfonic acids and alpha-, beta- and gamma-chloro and bromo-substituted carboxylic acids, e.g., hexadecylsulfonic acid, didodecylnaphthalene disulfonic acid, alpha-bromo lauric acid, beta, beta-dichloro decanoic acid and gamma dibromo octanoic acid, etc.

The amines of said invention are preferably primary, secondary and tertiary amines singly or in mixtures and characterized by having at least 10, and preferably at least 14, carbon atoms and at least one hydrophobic group. Such commercially available amines as Primene JM-5, and Primene JM-T, which are primary aliphatic amines in which the nitrogen atom is bonded directly to a tertiary carbon atom, and which commercial amines are sold by Rohm and Haas chemical Co.; Amberlite LA-1 and Amberlite LA-2, which are secondary amines sold by Rohm and Haas; Alamine 336, a tertiary thcaprylyl amine (TCA) and Alamine 304, a tertiary trilaurylamine (TLA), both sold by Cognis., can be used in the processes of said invention, as well as other well-known and available amines, including, e.g., those secondary and tertiary amines listed in U.S. Pat. No. 3,458,282.

The carrier solvents of said invention can be chosen from a wide range of organic liquids known to a person skilled in the art, which can serve as solvents for said acid-amine active components and which provide for greater ease in handling and extracting control. Said carrier solvents can be unsubstituted or substituted hydrocarbon solvents in which the organic acid and amine are known to be soluble and which are substantially water-insoluble, e.g., kerosene, mineral spirits, naphtha, benzene, xylene, toluene, nitrobenzene, carbon tetrachloride, chloroform, trichloroethylene, etc. Also higher oxygenated compounds such as alcohols, ketones, esters, ethers, etc., that may confer better homogeneity and fluidity and others that are not acids or amines, but which may confer an operationally useful characteristic, can also be included.

In the process of said invention, the essential operating extractant is believed to be the amine, balanced by a substantially equivalent amount of a strong organic acid. An excess of acid acts as a modifier of the system, and so does an excess of amine, which obviously will be present as salts of acids present in the system. These modifiers are useful in optimization of the extractant, but are not essential.

Thus, as stated, the molar ratio between the two foregoing active constituents lies between 0.5 to 2 and 2 to 0.5, and preferably between about 0.5 to 1 and 1 to 0.5.

The process as exemplified in the U.S. Pat. No. 4,291,007 patent was especially useful for use with acids such as nitric acid; however, the process as defined therein wherein the acid is recovered by backwashing is not practical or commercially viable for obtaining concentrated hydrochloric acid from dilute hydrochloric acid.

According to the invention described and claimed in PCT/IL2008/000278, it was surprisingly found that HCl can be distilled out of such an HCl-loaded extractant phase at temperatures below 250° C. without noticeable solvent decomposition.

Thus, said PCT/IL2008/000278 specification describes and claims a process for the recovery of HCl from a dilute solution thereof, comprising: a) bringing a dilute aqueous HCl solution into contact with a substantially immiscible extractant, said extractant comprising:

1) an oil soluble amine, which amine is substantially water-insoluble, in both free and salt forms;

2) an oil soluble organic acid, which acid is substantially water-insoluble, in both free and salt forms; and

3) a solvent for the amine and organic acid; whereupon HCl selectively transfers to said extractant to form an HCl-carrying extractant; and b) distilling HCl from said separated HCl-carrying extractant to form gaseous HCl and HCl depleted extractant.

In Israeli Patent Application 190,703, there is described and claimed a process for the recovery of HCl from a dilute solution thereof, comprising: a) bringing a dilute aqueous HCl solution into contact with a substantially immiscible extractant, said extractant comprising:

1) an oil soluble amine which amine is substantially water insoluble both in free and in salt form;

2) an oil soluble weak organic acid having a pka above 3 which acid is substantially water insoluble both in free and in salt form; and

3) a solvent for the amine and organic acid; whereupon HCl selectively transfers to said extractant to form an HCl-carrying extractant; and b) treating said HCl-carrying extractant to obtain gaseous HCl.

In Israeli Patent Application 190,704 there is described and claimed a process for the recovery of HCl from a dilute solution thereof, comprising: a) bringing a dilute aqueous HCl solution into contact with a substantially immiscible extractant, said extractant comprising:

1) an oil soluble amine, which amine is substantially water insoluble both in free and in salt form;

2) an oil soluble organic acid, which acid is substantially water insoluble both in free and in salt form; and

3) a solvent for the amine and organic acid; whereupon HCl selectively transfers to said extractant to form an HCl-carrying extractant; and b) treating said HCl-carrying extractant to obtain a mixture comprising HCl and a hydrocarbon in vapor phase for conveying the HCl from said extractant phase and for obtaining gaseous HCl.

The relevant descriptions and teachings of Israeli patent applications number IL190,703 and IL190,704 are incorporated herein by reference.

As will be realized, the process of the present invention is based on a modification and improvement of the process described in said PCT/IL2008/000278 specification and in said two later Israeli specifications, in that it utilizes the extraction and distillation steps thereof for the production of HCl gas from readily available chloride salts according to the equation:

MCl_{aq}+HX_{aq}+EXTRACTANT_{org}→MX_{aq}+EXTRACTANT.HCl_{org}

EXTRACTANT.HCl_{org}→EXTRACTANT_{org}+HCl_{gas}

The sum of these two stages:

MCl_{aq}+HX_{aq}→MX_{aq}+HCl_{gas}

wherein in these formulas, M represents the cation of the product salt and X the anion of that product salt.

As stated hereinbefore with reference to U.S. Pat. No. 4,291,007:

(1) “The strong organic acids envisioned for use in the extractant phase of said invention were organic acids which may be defined and characterized as follows: When 1 mol of the acid in a 0.2 molar or higher concentration is contacted with an equivalent amount of 1N NaCl, the pH of the sodium chloride solution decreases to below 3.

(2) Especially preferred for use in said invention were strong organic acids selected from the group consisting of aliphatic and aromatic sulfonic acids and alpha-, beta- and gamma-chloro and bromo-substituted carboxylic acids, e.g., hexadecylsulfonic acid, didodecylnaphthalene disulfonic acid, alpha-bromo lauric acid, beta, beta-dichloro decanoic acid and gamma dibromo octanoic acid, etc.”

In contradistinction to the teachings of said prior art patent, and the expectations from the above observations, it was surprisingly observed that weak acids, having a pKa above 3 and even very weak acids such as aliphatic carboxylic acids, can provide for effective stripping of part or the whole of HCl carried in an extractant of which the ABC extractant couples a weak acid with an amine.

Stated differently, weak acids such as carboxylic acids were not considered of interest in the practice of the invention as described in U.S. Pat. No. 4,291,007 or even as described in more recent application PCT/IL2008/000278, as constituents of ABC extractants or as constituents of extractants for HCl. Such extractants, when equilibrated with an aqueous HCl phase provide for powerful distribution in favor of the extractant, which distribution is only marginally affected by temperature. Stripping i.e.

distribution of HCl at higher temperatures in favor of the gas phase that generally parallels the distribution in favor of the aqueous phase was naturally expected to be ineffective in case of weak acids as component of ABC extractants. Surprisingly it has now been found that this parallelism breaks in the case of carboxylic acids and similar weak acids having a pKa above 3 and that effective stripping obtains. Furthermore, the effective extraction of HCl from an aqueous phase, which results in high loading of the extractant, provides for an economically beneficial reduction of the amount of extractant required per unit of HCl.

Thus in contradistinction to the definition of strong organic acids presented in U.S. Pat. No. 4,291,007, the weak organic acids envisioned for use in the extractant phase of the present invention are organic acids which may be defined and characterized as follows: when 1 mol of the acid in a 0.2 molar or higher concentration in an organic solvent is contacted with an equivalent amount of NaCl in 1 N aqueous solution, the pH of the sodium chloride solution is higher than about 4 more preferably higher than about 5.

Thus a weak acid according to the present invention, e.g. carboxylic acid such as lauric acid, when tested according to the above definition, reduces pH to about 6.

With regard to the pKa values of the acids mentioned in U.S. Pat. No. 4,291,007 as opposed to those envisioned for use in the present invention, said patent refers to—
“aromatic sulfonic acids” e.g. Naphtalenesulfonic acid the pKa is 0.17; and
“alpha-, beta- and gamma-chloro and bromo-substituted carboxylic acids”.

The following table sets forth the pKa values of these acids as opposed to those of the present invention:

TABLE 2

| Acid | pKa |
|---|---|
| alpha-bromo-butyric | 2.97 |
| 3,6-dichlorophtalic | 1.46 |
| Whereas typical values for the (surprisingly observed) | |
| weak acids such as unsubstituted carboxylic acids- | |
| Caproic | 4.88 |
| Caprylic | 4.90 |
| Lauric | 4.92 |

Such, the weak acids measure 2 or more pKa units higher than the acids previously described and claimed, corresponds to two orders of magnitude lower acidity.

Thus, it was surprisingly observed that weak acids, even very weak acids such as aliphatic carboxylic acids can provide for effective stripping of part or the whole of HCl carried in an extractant of which the ABC extractant couples a weak acid with an amine.

It is to be noted that the term “carrier solvent” as used herein is intended to denote the solvent component of the extractant used in the present invention.

The amines of the present invention are preferably primary, secondary and tertiary amines singly or in mixtures and characterized by having at least 10, preferably at least 14, carbon atoms and at least one hydrophobic group. Such commercially available amines as Primene JM-5, and Primene JM-T, which are primary aliphatic amines in which the nitrogen atom is bonded directly to a tertiary carbon atom, sold by Rohm and Haas Chemical Co.; Amberlite LA-1 and Amberlite LA-2, which are secondary amines sold by Rohm and Haas; Alamine 336, a tertiary tricaprylyl amine (TCA) and Alamine 304, a tertiary trilaurylamine (TLA), both sold by Cognis, Inc., can be used in the processes of the present invention, as well as other well known and available amines including, e.g., those secondary and tertiary amines listed in U.S. Pat. No. 3,458,282. According to a preferred embodiment, tris(2-ethyl hexyl) amine is used as an amine of the ABC extractant of the present invention.

The term “pH half neutralization (pHhn),” as used herein refers to the pH of an aqueous solution, which is in equilibrium with the extractant carrying HCl at an HCl-to-amine molar/molar ratio of 1:2.

According to other aspects of the invention, carbohydrates are formed by hydrolysis of polysaccharides such as cellulose and hemicellulose as found in lignocellulosic material, such as wood, sugarcane bagasse, straw and switch grass. Hydrolysis is conducted by contacting lignocellulosic material with HCl solution. HCl is not consumed in the process, but rather acts as a catalyst. The product of hydrolysis, also referred to as an hydrolyzate is an aqueous solution comprises carbohydrates and HCl. According to the process of the present invention, the hydrolyzate is treated for the separation of carbohydrates from HCl. The separated carbohydrates could then be used for various applications, e.g. as fermentation feedstock, while the separated acid is preferably reused for the hydrolysis.

Preferably, the acid used for hydrolysis is relatively pure in the sense that it is not a mixture of two acids. Working with such mixture, e.g. a mixture of HCl and H₂SO₄, would increase the cost of the production of the carbohydrates and add complications. Thus, recovery and recycle of such acid mixture is more expensive, e.g. not enabling the use of relatively low cost HCl evaporation. Furthermore, it is also difficult to maintain the ratio between the acids through repeated uses of the acid mixture, which could changes the conditions as the process continues and complicates control and optimization of process conditions, the acid is a catalyst within the hydrolysis therefore it is not consumed in the hydrolysis process. Having sulfate and phosphate ions in the hydrolyzing acid may also lead to the formation of water-immiscible salts, e.g. gypsum, which further complicates acid recovery and contaminate the product carbohydrate. Thus, hydrolysis with HCl, that is essentially free of sulfate and phosphate anions and recovery of such pure HCl for further use, is of high importance.

The recovered hydrochloric acid is reused for the hydrolysis of polysaccharides to carbohydrates. Various methods are known for the recovery of HCl from aqueous solutions and are also applicable for its recovery from the hydrolyzate formed on the hydrolysis of polysaccharides. In case the acid concentration in the hydrolyzate is high enough, part of the acid is recovered by distillation. Nevertheless, HCl and water have an azeotrope at about 22%. Recovery of all the HCl by distillation requires essentially drying the carbohydrates in the hydrolyzate and relatively high temperatures at which those carbohydrates start to degrade.

Solvent extraction was found to provide efficient separation of the non-distilled HCl. The extractant used needs to be selected such that the acid is extracted efficiently and selectively, i.e. with no carbohydrates and no other acids. Furthermore, the extracted acid needs to be recovered from the HCl-containing extractant in an acid form at a concentration high enough to enable reformation of the hydrolyzing acid solution at the required concentration, which according to a preferred embodiment is higher than 37% wt., and more preferably higher than 40% wt.

HCl recovery yield according to the process of the preset invention is high, preferably greater than 95%, and more preferably greater than 97%. Yet, some HCl loss could not be avoided and acid makeup is required. As indicated, that make up should be of pure HCl, rather than a mixture of HCl and another acid. There is the possibility of purchasing an HCl solution for that makeup. According to the process of the present invention, the makeup acid is formed by reacting a chloride salt (MCl) and another acid (HX), which acid is weaker and or more hydrophilic than HCl. Acidic salts of such acids are also suitable. Thus, according to an embodiment of the invention, such MCl is combined with such HX or acidic salt thereof to form an aqueous solution containing chloride anions and X anions, protons and the cations (M) of MCl. The formed solution is contacted with an extractant that selectively extracts HCl from the mixture to form HCl-carrying extractant and a chloride depleted aqueous solution. Those are separated and the aqueous solution is preferably treated for the recovery of MX, as explained above. The separated HCl-carrying extractant is then treated for the recovery of HCl therefrom, forming an HCl solution that could be reused in hydrolysis.

As indicated, the recovered HCl for reuse in hydrolysis should be substantially pure as hereinafter defined, especially low in phosphate and sulfate, so that extraction from the aqueous solution formed on the combination of MCl and HX will be selective, preferably extracting very little of HX along with HCl. The recovered pure HCl is reused in the hydrolysis of carbohydrate to form the hydrolyzate and that step is followed by HCl recovery of HCl from the hydrolyzate.

Thus in an aspect of the present invention, there is also provided a process for the production of carbohydrates comprises: a. combining an alkali or an ammonium salt in solution with a water soluble acid or an acidic salt having an acidity weaker than hydrochloric acid; b. bringing said solution into contact with a substantially immiscible extractant, said extractant comprising:

1) an oil soluble amine, which amine is substantially water insoluble both in free and in salt form;

2) an oil soluble weak organic acid which acid is substantially water insoluble both in free and in salt form; and

3) a carrier solvent for the amine and organic acid; whereupon HCl selectively transfers to said extractant to form an HCl-carrying extractant and a chloride depleted solution containing the anion of said weak acid and the alkali or ammonium cation; c. separating said HCl-carrying extractant from said chloride-depleted aqueous solution; d. recovering a high-purity HCl from said HCl-carrying extractant to form a high-purity HCl stream and an HCl-depleted extractant; e. hydrolyzing a lignocellulosic material with said high-purity HCl stream to form a carbohydrates-comprising and an HCl-comprising hydrolyzate; and f. separating HCl from the carbohydrates within said hydrolyzate.

According to yet further aspect of the invention, pure HCl is used for the hydrolysis of the polysaccharides to form the HCl-comprising hydrolyzate, and HCl is recovered from that hydrolyzate by means of solvent extraction. A first extractant is combined with the hydrolyzate whereupon pure HCl transfers into it to form an HCl-depleted hydrolyzate and a first HCl-carrying extractant. The HCl-depleted hydrolyzate and the first HCl-carrying extractant are separated to form a separated HCl-depleted hydrolyzate comprises the formed carbohydrates and a separated first HCl-carrying extractant. The separated first HCl-carrying extractant is then treated to recover pure HCl therefrom and to regenerate the first extractant. The recovered pure HCl is reused to hydrolyze the polysaccharides.

According to that aspect there is some HCl loss and an HCl makeup is required. Such HCl makeup is formed by means of combing MCl and HX as described above and separating HCl from that aqueous solution by extraction. Such separating is conducted by combining the aqueous solution with a second extractant whereupon pure HCl transfers selectively to the extractant to form a chloride depleted aqueous solution and a second HCl-carrying extractant. The chloride depleted aqueous solution is then separated from the second HCl-carrying extractant and the latter is treated to recover pure HCl therefrom and to regenerate the second extractant. The recovered pure HCl is reused to hydrolyze the polysaccharides.

According to an embodiment, the first extractant used for the recovery of HCl from the hydrolyzate has a similar composition as the second extractant used for the production of the HCl makeup. In a preferred embodiment the two extractants have the exact same composition. According to that embodiment, the first HCl-carrying extractant has a composition similar to that of the second HCl-carrying extractant, particularly in terms of the extractant components and the weight/weight ratios between these components. There could however be differences within the HCl concentrations therein and within the content of some other components, e.g. the concentration of co-extracted water. According to a further preferred embodiment, rather than separately treating the first and the second HCl-carrying extractants for the HCl-recovery, the two are combined to form a combined extract and that combined extract is treated for the recovery of HCl and for the regeneration of the extractant. The recovered HCl, comprises the HCl extracted from the hydrolyzate and the HCl recovered in the makeup production step, is used for hydrolysis of the polysaccharides to form another hydrolyzate.

The inventors have found that the ABC extractants as described above are efficient and selective in the extraction of pure HCl from the hydrolyzate of the polysaccharides and that the extractant enables the recovery of the extracted HCl at a purity and concentration sufficiently high to be reused for the hydrolysis of the polysaccharides. The inventors have surprisingly found that the ABC extractant is also efficient in the separation of HCl from the aqueous solution formed from the production of HCl makeup from the mixture formed by combining MCl and HX. Furthermore, that extractant shows high selectivity in extraction from that aqueous solution, such HCl is extracted with essentially no HX. Therefore, the HCl formed on recovery from that extractant is concentrated enough and pure enough for use in the hydrolysis of the polysaccharides. It was further found that the same composition of the ABC extractant can be used for both the recovery from the hydrolyzate and the HCl makeup operation. The surprising finding that a given extractant combines efficient, reversible and selective recovery from the carbohydrates-comprising polysaccharides hydrolyzate and efficient, reversible and selective HCl recovery of HCl from the MX-comprising makeup production solution enables an economically attractive process of carbohydrates production from polysaccharides by hydrolysis with a pure HCl solution, recovery of the HCl for reuse, production of HCl makeup and all that with the same extractant and a single HCl recovery from the HCl-carrying extract.

Thus, in an aspect of the present invention there is provided a process for the production of carbohydrates comprises:

a. hydrolyzing a lignocellulosic material with high-purity HCl stream to form a carbohydrates-comprising and an HCl-comprising hydrolyzate;

b. bringing said hydrolyzate into contact with a substantially immiscible extractant, said extractant comprises:
1) an oil soluble amine, which amine is substantially water insoluble both in free and in salt form;
2) an oil soluble weak organic acid which acid is substantially water insoluble both in free and in salt form; and
3) a carrier solvent for the amine and organic acid;
whereupon HCl selectively transfers to said extractant to form a first HCl-carrying extractant and an HCl-depleted hydrolyzate;

c. separating said first HCl-carrying extractant from said hydrolyzate;

d. combining an alkali or ammonium salt within solution with a water soluble acid or acidic salt having an acidity weaker than hydrochloric acid;

e. bringing said solution into contact with said extractant whereupon HCl selectively transfers to said extractant to form a second HCl-carrying extractant and a chloride-depleted solution containing the anion of said weak acid and the alkali or ammonium cation;

f. separating said second HCl-carrying extractant from said chloride-depleted aqueous solution;

g. combining at least a portion of said first HCl-carrying extractant and a portion of said second HCl-carrying extractant to form a combined extract;

h. separating a high-purity HCl from said combined extract to form a separated high-purity HCl and a regenerated extractant; and

i. using said high-purity HCl for hydrolyzing said lignocellulosic material.

According to an embodiment of the invention, said regenerated extractant is then preferably divided into two portions. The first of those two portions is used for the recovering and separating of HCl from that other hydrolyzate and the second portion is used for generating of additional HCl makeup. According to an embodiment, the first portion is greater than the second portion, e.g. greater by 3 to 20 folds.

As used herein, the term pure HCl refers to both HCl gas and HCl aqueous solutions. The term “pure” herein means according to various embodiments, purity greater than 90%, preferably greater than 95%, more preferably greater than 98%, and most preferably greater than 99% of the total acid within that stream in molar ratio.

According to another embodiment, pure means HCl to (H₂SO₄+H₃PO₄) molar ratio of greater than 10, preferably greater than 20, more preferably greater than 40, and most preferably greater than 50.

Any method of acid recovery from the first HCl-carrying extractant, the second HCl-carrying extractant and extract is suitable. According to a preferred embodiment, recovery uses at least one of distillation to form a gaseous HCl stream and multiple-stage counter-current back extraction with water or with an aqueous solution to form an aqueous solution of HCl, also referred to as back-extract. According to a preferred embodiment, recovery uses multiple-stage counter-current back extraction with water wherein the number of stages is at least 3, preferably at least 4, more preferably at least 5. According to another preferred embodiment, the HCl concentration in the back-extract is at least 15% wt, preferably at least 20% wt, and more preferably at least 22% wt. According to still another embodiment, recovery uses counter-current back-extraction and said counter-current back-extraction is conducted at a temperature of at least 20° C. greater than the temperature of said combining with the hydrolyzate.

While the invention will now be described in connection with certain preferred embodiments in the following examples so that aspects thereof may be more fully understood and appreciated, it is not intended to limit the invention to these particular embodiments. On the contrary, it is intended to cover all alternatives, modifications and equivalents as may be included within the scope of the invention as defined by the appended claims. Thus, the following examples which include preferred embodiments will serve to illustrate the practice of this invention, it being understood that the particulars shown are by way of example and for purposes of illustrative discussion of preferred embodiments of the present invention only and are presented in the cause of providing what is believed to be the most useful and readily understood description of formulation procedures as well as of the principles and conceptual aspects of the invention.

### EXAMPLES

### Example 1

52 gr commercial 98% phosphoric acid were dissolved in 200 ml water, then 38 gr fertilizer grade KCl, potash, were added. They dissolved with the help of a little stirring. The resulting, slightly clouded solution was contacted three successive times with 500 gr, each time, of an extractant of a composition by weight: 14.6% n-octanoic acid; 35.3% tris(2-ethylhehyl)amine and 50.1% carrier solvent consisting of a hydrocarbons distillate boiling in the range 180/210° C. at atmospheric pressure—and these successive portions were combined. The concentration of chloride in the extracted aqueous solution was below 0.01% indicating a practically complete conversion of the potassium chloride to potassium phosphate. The combined extractant was boiled under vacuum at 160° C. whereby HCl was distilled and collected in 0.1 N NaOH for titration. 16.2 grs of HCl were titrated—practically corresponding to complete conversion.

### Example 2

HCl was mixed with sulfuric acid solutions to form aqueous solutions containing protons and ions of potassium chloride and sulfate. These solutions were contacted in vials with the extractant of Example 1. The vials were shaken at RT. Samples were taken to analysis and the results are presented in the following table:

| | | | Org. phase composition | | |
|---|---|---|---|---|---|
| Aq. phase composition | | | | H₂SO₄ | |
| K₂SO₄ | Cl | H₂SO₄ | Cl | calculated | selectivity |
| Mol/kg | mol/kg | mol/kg | mol/kg | mol/kg | HCl/SO₄ |
| 0.74 | 0.36 | 0.0120 | 0.227 | 0.0013 | 369 |
| 0.78 | 0.27 | 0.0090 | 0.052 | 0.001 | 135 |
| 0.79 | 0.17 | 0.0070 | 0.014 | 0.0005 | 129 |
| 0.71 | 0.41 | 0.015 | 0.27 | 0.002 | 233 |

The results show the very high selectivity of the extractant towards HCl when compared with extraction of sulfuric acid.

It will be evident to those skilled in the art that the invention is not limited to the details of the foregoing illustrative examples and that the present invention may be embodied in other specific forms without departing from the essential attributes thereof, and it is therefore desired that the present embodiments and examples be considered in all respects as illustrative and not restrictive, reference being made to the appended claims, rather than to the foregoing description, and all changes which come within the meaning and range of equivalency of the claims are therefore intended to be embraced therein.

What is claimed is;:

1. A process for the co-production of gaseous HCl and a salt product comprising a cation and an anion, which process comprises the steps of
a. providing an aqueous solution comprising protons, chloride anions, and cations and anions of said salt product;
b. bringing said solution into contact with a substantially immiscible extractant, said extractant comprising:
1) an oil soluble amine, which amine is substantially water insoluble both in free and in salt form; and
2) a carrier solvent for the amine;
whereupon HCl selectively transfers to said extractant to form an HCl-carrying extractant and a chloride depleted aqueous solution containing said salt product;
c. separating said HCl-carrying extractant from said chloride-depleted aqueous solution; and
d. distilling HCl from said separated HCl-carrying extractant to form gaseous HCl and HCl depleted extractant; and wherein said extractant further comprises an oil soluble organic acid, which acid is substantially water insoluble both in free and in salt form and wherein the pKa of the organic acid is above 3.

2. A process according to claim 1, wherein said extractant has a pHhn of less than 3.

3. A process according to claim 1, further comprising;
e. recovering said salt product of said separated chloride-depleted aqueous solution of step (c), wherein said recovery comprises crystallization to form crystals of said salt and a mother liquor and wherein said mother liquor is used for said providing of step (a).

4. A process according to claim 1, wherein step (a) is carried out by combining a chloride salt of said cation with a compound selected from an acid of said anion and an acidic salt of said anion in the presence of water.

5. A process according to claim 1, wherein said cation is selected from the group consisting of alkali ions and ammonium.

6. A process according to claim 1, wherein said anion is selected from the group consisting of anions of acids that are weaker than HCl and anions that are more hydrophilic than chloride anions.

7. A process according to claim 1, wherein said anion is selected from the group consisting of phosphate, nitrate, sulfate, carbonate and bicarbonate.

8. A process according to claim 1, wherein said distilling is at a temperature lower than 250° C.

9. A process according to claim 1, wherein said gaseous HCl comprises at least 90% of the chloride in said provided solution of step (a).

10. A process for the production of gaseous HCl from an alkali or ammonium chloride salt comprising:
a. combining an alkali or ammonium salt in solution with a water soluble acid or acidic salt having an acidity weaker than hydrochloric acid;
b. bringing said solution into contact with a substantially immiscible extractant, said extractant comprising:
1) an oil soluble amine, which amine is substantially water insoluble both in free and in salt form;
2) an oil soluble weak organic acid which add is substantially water insoluble both in free and in salt form; and
3) a carrier solvent for the amine and organic acid;
whereupon HCl selectively transfers to said extractant to form an HCl-carrying extractant and a chloride depleted solution containing the anion of said weak acid and the alkali or ammonium cation; and
c. distilling HCl from said HCl-carrying extractant to form gaseous HCl and HCl depleted extractant.

11. A process according to claim 10, further comprising:
d. recovering the salt of said anion of said weak acid and the alkali or ammonium cation from the chloride depleted solution of step (b).

12. A process according to claim 11, wherein phosphoric acid is combined in solution with potassium chloride and there is obtained substantially pure gaseous HCl and potassium phosphate.

13. A process according to claim 10, wherein said extractant has a pHhn of less than 3.

14. A process according to claim 10, wherein the pKa of the organic acid is above 3.

15. A process for the production of carbohydrates comprising:
a. hydrolyzing lignocellulosic material with high-purity HCl stream to form a carbohydrates-comprising and HCl-comprising hydrolyzate;
b. bringing said hydrolyzate into contact with a substantially immiscible extractant, said extractant comprising:
1) an oil soluble amine, which amine is substantially water insoluble both in free and in salt form;
2) an oil soluble weak organic acid which acid is substantially water insoluble both in free and in salt form; and
3) a carrier solvent for the amine and organic acid;
whereupon HCl selectively transfers to said extractant to form a first HCl-carrying extractant; and an HCl-depleted hydrolyzate;
c. separating said first HCl-carrying extractant from said hydrolyzate;
d. combining an alkali or ammonium salt in solution with a water soluble acid or acidic salt having an acidity weaker than hydrochloric acid;
e. bringing said solution into contact with said extractant; whereupon HCl selectively transfers to said extractant to form a second HCl-carrying extractant; and a chloride-depleted solution containing the anion of said weak acid and the alkali or ammonium cation;
f. separating said second HCl-carrying extractant from said chloride-depleted aqueous solution;
g. combining at least a portion of said first HCl-carrying extractant and a portion of said second HCl-carrying extractant to form a combined extract;
h. separating high-purity HCl from said combined extract to form separated high-purity HCl; and
i. using said high-purity HCl for hydrolyzing lignocellulosic material.
